(19) United States
(12) Patent Application Publication
(10) Pub. No.: US 2006/0292584 A1
(43) Pub. Date: Dec. 28, 2006

(54) Compositions and methods for accomplishing nucleotide depletion

(75) Inventors: Pei-Chung Hsieh, Topsfield, MA (US); William Jack, Wenham, MA (US); Lucia Greenough, Ipswich, MA (US)

Correspondence Address: HARRIET M. STRIMPEL; NEW ENGLAND BIOLABS, INC., 240 COUNTY ROAD, IPSWICH, MA, 01938-2723, US

(73) Assignee: New England Biolabs, Inc., Ipswich, MA (US)

(21) Appl. No.: 11/210,148

(22) Filed: Aug. 23, 2005

Related U.S. Application Data
(60) Provisional application No. 60/604,141, filed on Aug. 24, 2004.

(51) Int. Cl. C12Q 1/68; C12P 19/34

(52) U.S. Cl. 435/6; 435/91.2

(57) ABSTRACT

Methods and compositions are provided that achieve depletion of a nucleotide pool by means of a phosphate-transferring enzyme such as a nucleoside phosphate or a polyphosphate glucokinase. Depletion of a nucleotide pool using a nucleoside kinase may additionally utilize a phosphotransferase in a second phosphate-transferring reaction.

### CROSS REFERENCE

This application claims priority to provisional application Ser. No. 60/604,141 filed Aug. 24, 2004, herein incorporated by reference.

### BACKGROUND

Currently, numerous molecular biology applications utilize nucleotide incorporation for DNA analysis, for example, DNA sequencing and single nucleotide polymorphism (SNP) analysis. Typical compounds included in a DNA analysis are: (1) a template nucleic acid; (2) a nucleic acid primer that hybridizes to that template; and (3) nucleotide triphosphates that are used to extend the annealed primer in a template-directed action by (4) a nucleic acid polymerase. When the amount of template is limited it is desirable to increase the template concentration prior to DNA analysis. This is achieved by a template amplification step that employs reagents similar to those used in DNA analysis. However, DNA analysis can be compromised if those similar amplification reagents carry-over into the analysis reactions.

For example, a PCR reaction is frequently used to amplify the template, a reaction that requires addition of single-stranded primers and deoxynucleoside triphosphate (dNTPs). Deoxynucleoside triphosphates have the potential to interfere with downstream reactions. For example, Sanger-type DNA sequencing employs a substrate pool containing both dNTPs and nucleotide analogs that act as DNA synthesis terminators. The ratio of dNTPs to terminators determines the frequency of terminator incorporation, and is a critical feature in defining the size range of products produced by the reaction. The presence of unknown amounts of dNTPs from an amplification reaction will thus adversely affect DNA sequence analysis.

One approach to eliminating interference from amplification reagents is to remove primers and dNTPs from amplification products by physical means. Examples of such methods are: (1) gel electrophoresis to separate reaction products, with selective elution of the desired double-stranded DNA amplification product; (2) gel filtration columns that separate the amplification product from the smaller primers and dNTPs based on molecular weight/shape; and (3) affinity resins that selectively retain the larger amplification products, which can then be selectively eluted. However, such methods require a number of manipulations that take additional time and effort, and often reduce product yields.

Alternatively, dNTPs can be converted into forms that do not interfere with subsequent reactions using phosphatases (see for example U.S. Pat. Nos. 5,741,676, 5,756,285 and 6,379,940). Since nucleoside triphosphates are requisite substrates for polymerases, the removal of one or more phosphates from the dNTP or ribonucleoside triphosphate (NTP) obviates their ability to function as polymerization substrates. One problem associated with the use of phosphatases is their removal before subsequent reactions.

Because of the limitations of present methods, it is desirable to find an improved cost effective approach for inactivation of unwanted deoxynucleotides in molecular biology reactions.

### SUMMARY

In an embodiment of the invention, a method is provided of depleting a nucleotide pool, that includes the steps of: (a) adding to the nucleotide pool, a primary phosphate acceptor, and a phosphate-transferring enzyme, where the phosphate-transferring enzyme is exemplified by a nucleoside kinase or a polyphosphate glucokinase; and (b) permitting the conversion of dNTP to deoxynucleoside diphosphate (dNDP) so as to deplete the nucleotide pool. Once depleted by more than 85%, the primary enzyme may be substantially inactivated by heat, for example, at a temperature between 700 and 100° C. Heat inactivation may be accomplished within 60 mins after raising the temperature.

In an example of the method, where the primary enzyme is a nucleoside kinase such as nucleoside 5′diphosphate kinase, the method may further use a secondary enzyme such as a phosphotransferase or a lyase where the secondary enzyme dephosphorylates the phosphate acceptor so as to modify the equilibrium of the reaction with the primary enzyme in favor of dephosphorylation of the dNTP or NTP in the nucleotide pool. Where the secondary enzyme is a phosphotransferase, the reaction may further utilize a secondary phosphate acceptor, the acceptor depending on the phosphotransferase employed.

In an embodiment of the invention, a reaction mixture is provided for depleting a nucleoside triphosphate pool, where the mixture contains a gamma phosphate-transferring enzyme such as a nucleoside kinase or polyphosphate glucokinase for removing a phosphate from a dNTP or NTP in a nucleotide pool and a primary nucleoside phosphate acceptor, for example, a dNTP or a ribonucleoside diphosphate or a monosaccharide, for example ATP. If the phosphate-transferring enzyme is a nucleoside kinase, a second enzyme may be used in the reaction mixture, for example, phosphotransferase or lyase. The phosphotransferase or lyase catalyzes removal of the phosphate from the primary nucleoside phosphate acceptor so as to drive the equilibrium reaction catalyzed by the nucleoside kinase toward depletion of the nucleotide pool. The mixture may additionally contain a second acceptor and may also contain a nuclease.

In an embodiment of the invention, a nucleotide depletion reagent is provided that is capable of gamma phosphate transfer from a dNTP or NTP to a phosphate acceptor so as to reduce the concentration of dNTPs or NTPs in the pool by at least 85%, at least 80% of the depletion reagent being denatured at a temperature of less than 100° C. for an incubation period of less than 60 minutes.

For example, the nucleotide depletion reagent may be a nucleoside kinase such as nucleoside 5′diphosphate kinase, or a polyphosphate glucokinase, and further includes a primary acceptor. Where the nucelotide depletion reagent is a nucleoside kinase, a secondary enzyme may be added, for example, a phosphotransferase or lyase. If the second enzyme is a phosphotransferase, a secondary acceptor is also preferably added to the nucleotide depletion reagent.

In a further embodiment of the invention, a kit is provided which contains a nucleotide depletion reagent or a reaction mixture such as described above and optionally instructions for use.

### FIGURES

FIG. 1 shows a 10-20% of Tris-glycine SDS-PAGE on which purified polyphosphate glucokinase is displayed. Lane M, protein marker (New England Biolabs, Inc., Ipswich, Mass., catalog #P7702); lane 1, 2 μl of crude extract; lane 2, 2 μl of amylose column elutant; lane 3, 6 μl of amylose column eluant. The arrow indicates the position of the maltose-binding protein (MBP)-polyphosphate glucokinase (PPGK) fusion protein.

FIG. 2 shows an enzymatic degradation reaction for dNTPs by polyphosphate glucokinase. Reactions were performed as described in Example II. Curves indicate dATP (□), dCTP (◯), dGTP (⋄) or TTP (▴).

FIG. 3 shows conversion of dCTP to dCDP in the presence of polyphosphate glucokinase.

FIG. 4 shows heat inactivation of PPGK.

FIG. 5 shows that PPGK degrades dATP in a time-dependent manner.

FIG. 6 shows that a mixture of nucleoside 5′diphosphate kinase (NDPK)/hexokinase degrades dCTP in a time-dependent manner.

FIG. 7 shows that sequencing of PCR reactions is aided by pre-treatment with Exonuclease I and PPGK (top line untreated—SEQ ID NO:5 and bottom line pre-treated—SEQ ID NO:6).

### DESCRIPTION

An improved method of inactivating dNTP or NTP pools prior to DNA or RNA analysis is provided in which the degradative reaction that relies on phosphatases is substituted with an alternative more cost effective phosphate transferring enzyme reaction or reactions.

The use of phosphate-transferring enzymes for reducing pools of dNTPs after DNA synthesis by DNA polymerases can also be used to reduce pools of NTPs after RNA synthesis Similarly, the skilled artisan will recognize that references to DNA polymerases could be readily expanded to other nucleic acid metabolic enzymes, including but not limited to terminal transferases and reverse transcriptases. The terms “deoxynucleoside triphosphate” or “dNTP” and “ribonucleoside triphosphates” or “NTP” are intended to include native nucleoside triphosphates as well as labeled or chemically modified dNTPs or NTPs, for example, methylated, biotinylated, halogenated or fluorescently labeled dNTPs or NTPs. A pool of dNTPs or NTPs may include all or a subset of the four different nucleotides.

Phosphate-Transferring Enzymes

Embodiments of the present methods and compositions utilize or incorporate an enzyme or enzyme combinations having one or more of the following properties:

(a) The ability to transfer the gamma phosphate group from dNTPs or NTPS, preferably with little discrimination between the different dNTPs (see for example, Morrison et al. Annual Review of Biochemistry 41:29-54 (1972)).

(b) retention of activity in buffers commonly used in amplification reactions. Examples of buffers used for PCR are (1) PCR buffer from Roche Applied Science, Basel, Switzerland: 10 mM TrisHCl (pH 8.3), 50 mM KCl, 2 mM MgCl₂; and (2) Thermopol Buffer from New England Biolabs, Inc., Ipswich, Mass.: 20 mM TrisHCl (pH 8.8), 10 mM KCl, 10 mM (NH₄)₂SO₄, 2 mM MgSO₄, 0.1% Triton X-100, 0.2 mg/ml BSA. Other recommended buffers can be found by consulting the enzyme supplier technical literature.

(c) the ability to be heat-inactivated following the phosphate-transferring reaction and prior to subsequent reactions.

The phosphate transfer may be achieved in one step or may involve more than one step, where a second or additional steps are used to increase the fraction of dNTPs from which phosphate groups are transferred, for example, by providing a kinetic environment that favors such transfer. Dephosphorylation reaction or reactions should preferably reduce the pool of dNTPs or NTPs by at least 85%, more preferably by at least 90%, more preferably at least 95%.

Nucleotide depletion can be functionally defined as the use of any enzyme capable of gamma phosphate transfer from a dNTP or NTP to a phosphate acceptor. Depletion is the result of reducing the concentration of dNTPs or NTPs in a pool by at least about 85%. The enzyme should be capable of at least 80% heat denaturation at a temperature of less than 100° C. for an incubation period of less than 60 mins at the denaturing temperature.

The suitability of any particular phosphate-transferring enzyme or enzymes can be established using a radioactive thin-layer chromatography assay described in Example II. This assay can be used to determine not only the suitability of candidate enzymes for the reactions described here, but also to test any putative improvements to kinetic characteristics, and suitability of the reaction buffer.

A phosphate-transferring reaction may be accompanied by removal of residual short single-stranded oligonucleotide primers from amplification mixtures using a nuclease. This nuclease reaction can be performed in conjunction with phosphate transfer or as a separate step. A preferred property of the nuclease is that it can selectively degrade the short oligonucleotide primers, which are single-stranded, while not degrading the amplified material, which is double-stranded. An example of a suitable nuclease is Exonuclease I.

In an embodiment of the invention, the phosphate-transferring reaction is achieved using a polyphosphate glucokinase (PPGK), which has been shown to cause dNTP or NTP depletion in one step.

PPGK is readily isolated from natural sources such as Actinomycetales (Hsieh, et al. Protein Exp. Purif. 4:76-84 (1993); Pepin, et al. J. Biol. Chem. 261: 4476-4480 (1986); Szymona and Szymoma Acta Microbiol. Pol. 28:153-160 (1979), Myxococcus coralloides D (Gonzalez, et al. D. Arch. Microbiol. 154:438-442 (1990)) from the bacterial parasite Bdellovibrio bacteriovorus (Bobyk, et al. Zentralbl Bakteriol Naturwiss 135(6):461-6 (1980)) and from the oligotrophic bacteria Renobacter vacuolatum (Kulaev and Vagabor Adv. Microb. Physiol. 24:83-117 (1983)). This family of enzymes can be used here to remove the gamma-phosphate from deoxyribonucleotides in a single reaction step transferring phosphate groups from a pool of dNTPs or NTPs to an acceptor substrate. This single enzyme will preferably react with all dNTPs or NTPs in the pool with similar efficiency regardless of whether they are dCTP, dATP, dTTP, dGTP, CTP, GTP, UTP or ATP, resulting in depletion of all dNTPs or NTPs in the pool. The reaction efficiently converts a large fraction of the dNTP pool into an inactive form (greater than 90%). For example, it is shown here that PPGK utilizes all four dNTPs as donor substrates in this reaction (Reaction 1), in the presence of glucose acting as acceptor
dNTP+D-glucose<−>dNDP+D-glucose-6-phosphate (Reaction 1)

In another embodiment, phosphate-transfer is achieved using an enzyme with nucleoside kinase activity (referred to here as a nucleoside kinase) that can be obtained from eukaryotic, archeal or prokaryotic cells. A nucleoside kinase can be used to deplete dNTP or NTP (Reaction 2) in a coupled reaction with at least one additional enzyme and acceptor (Reaction 3). The second reaction involving a second enzyme and second acceptor results in removal of a phosphate from ATP or GTP. (Reaction 3 exemplifies the first acceptor being ADP.) This reaction drives the equilibrium reaction catalyzed by the nucleoside kinase to favor formation of ATP. In the process ADP is regenerated and can once again be used by the nucleoside kinase in the primary phosphate-transferring reaction. The net reaction is illustrated in Reaction 4 for a dNTP but could similarly apply to an NTP. The net reaction is shown in Reaction 4.

The coupled reaction can be summarized as follows:
ADP+dNTP<->ATP+dNDP (Reaction 2)
ATP+second acceptor<->ADP+Second acceptor(P) (Reaction 3)
dNTP+second acceptor--->dNDP+second acceptor(P) (net Reaction 4)

The broad specificity of the above reaction is ideal for simultaneously depleting the dNTPs remaining after amplification.

Nucleoside kinases have a broad substrate specificity for all four dNTPs or NTPs, transferring the gamma phosphate from a variety of deoxy- and ribonucleoside triphosphates to a variety of deoxy- and ribonucleoside diphosphate acceptors. If the acceptor is ADP, the phosphorylated acceptor product is ATP. This broad substrate specificity can be used to inactivate a wide variety of dNTPs via conversion to dNDPs (Ray, et al. Curr Top Cell Regul 28: 343-357 (1992) and Mathews, Basic Life Sci. 31: 47-66 (1985)). Examples of enzymes with nucleoside kinase activity, include Pk (Sundin, et al. Mol Microbiol 28:965-979 (1996)), adenylate kinase (Lu, et al. Proc Natl Acad Sci USA 28:5720 5725 (1996)), and polyphosphate kinase (Kuroda, Proc Natl Acad Sci USA 28:439-442 (1997)).

The conversion of ADP to ATP is a very well understood reaction and occurs in many different reactions of cellular metabolism, such as cell respiration where dephosphorylation of ATP generates a major source of energy in a cell (see for example, H. R. Mahler and E. H. Cordes, Biological Chemistry, Harper & Row Publishers, Second Edition, New York, N.Y. pp. 337-384 (1971); A. L. Lehninger, Biochemistry, Worth Publishers, Inc., New York, N.Y., 2^{nd} ed. pp. 387-416 (1975); Kornberg, A. and Baker, T. A., in DNA Replication, 2nd ed., W.H. Freeman and Co., New York, N.Y. p. 68 (1992)). A source of nucleoside kinases, and enzymes suitable for a second reaction (for example, phosphotransferases E.C.2.7) (Fasman G. D. ed, 3rd ed., CRC Press, Cleveland, Ohio pp. 93-109 (1975)) that enhances in a favorable direction the kinetics of the first reaction can be obtained commercially, for example, from the SIGMA catalog (Sigma-Aldrich, St. Louis, Mo.).

An example of a nucleoside kinase is NDPK. This enzyme has an equilibrium constant that is near unity when transferring a gamma phosphate from a dNTP or NTP to an acceptor such as ADP, meaning that by itself, NDPK would have difficulty depleting dNTP pools to low levels. (see Reaction 2). To overcome this obstacle an additional coupled reaction can be employed, for example, one catalyzed by hexokinase. In this reaction, the secondary phosphate acceptor is glucose.
ATP+D-glucose<->ADP+D-glucose-6-phosphate (Reaction 5).

Unlike the reaction with NDPK, the reaction kinetics for hexokinase favor the products ADP and D-glucose-6-phosphate. Thus, low concentrations of reactants (i.e., ATP) are converted more efficiently into products (i.e., ADP). Inclusion of excess concentrations of D-glucose leads to an even higher production of ADP. By coupling the NDPK reaction to this second reaction, the equilibrium constant strongly favors product formation and the deficiency in nucleotide depletion with NDPK alone is overcome so that a significant fraction of dNTPs can be converted to dNDPs.

The net result of the two simultaneous coupled reactions is:
dNTP+glucose<->dNDP+D-glucose-6-phosphate (Reaction 6).

The hexokinase reaction is just one of many examples of a second enzyme that is effective at converting ATP back to ADP in the secondary reaction. Not only do phosphotransferases other than hexokinases utilize glucose as a phosphate acceptor but there are many different phosphotransferases known in the art that use a variety of different phosphate acceptors (see for example, Table I). In the presence of the phosphotransferase, the final levels of dNTP are reduced in comparison to the reaction with NDPK alone. Use of NDPK provides a useful bridge to enzymes that convert ATP to ADP in coupled nucleotide depletion reactions.

While the above example of a nucleoside kinase reaction for depleting a nucleotide pool utilizes two enzymes, additional embodiments may utilize more than two enzymes. For example, a first enzyme acceptor could inactivate a subset of the dNTP or NTP pool, and a second enzyme could then inactivate a different spectrum of dNTPs or NTPs from the pool, etc. Further efficiencies of dNTP or NTP depletion can also be achieved by using a third enzyme to convert or regenerate the second acceptor after phosphorylation.

Table 1 lists examples of phosphate acceptor molecules in addition to ADP that can be used with phosphotransferases in coupled secondary phosphate-transferring reactions with the primary nucleoside diphosphate transferase reaction.

In one embodiment, glycerol kinase catalyzes the transfer of the gamma-phosphate from ATP to glycerol (the acceptor), with the end products being ADP and glycerol-3-phosphate. This list is intended to illustrate potential secondary enzyme/acceptor combinations in coupled reactions with a nucleoside kinase and is not intended to be an exhaustive listing.

TABLE 1

| Phosphate Acceptor | Phosphotransferase |
|---|---|
| rADP | nucleoside diphosphate kinase |
| Monosaccharides, e.g. glucose | hexokinase |
| glycerol | glycerol kinase |
| D-glycerate | gycerate kinase |
| D-fructose | ketohexokinase |
| D-galactose | galactokinase |
| Pantetheine | pantetheine kinase |
| L-1-phosphatidyl-inositol | phosphatidylionsitol kinase |
| N-acetyl-D-glucosamine | N-acetyl-D-glucosamine kinase |
| Skikimate | shikimate kinase |
| nicotinamide adenine | NAD kinase |
| dinucleotide | |
| N-acetyl-glutamate | N-acetyl-glutamate kinase |
| Glucose | glycerol-3-phosphate-glucose |
| | phosphotransferase |

While the coupled enzyme reaction is described in terms of phosphate transfer, a reaction that hydrolyzes the phosphorylated acceptor can also be utilized to regenerate the acceptor. Such an action can be provided, for example, by lyases.

In one embodiment, either an initial or second reaction may utilize lyase in addition to or instead of a phosphotransferase. A lyase is an enzyme that catalyzes the addition of groups to double bonds, or vice versa. It is here included as an example of a phosphate-transferring enzyme although for lyases, the transferred phosphate may remain free and not coupled to an acceptor. The acceptor in the cases exemplified below is citrate, which becomes oxaloacetate, L-aspartate which becomes L-asparagine succinate which becomes succinyl CoA and glutamate which becomes L-gamma glutamylcysteine.

For example, ATP citrate lyase catalyzes the reaction:
Citrate+ATP<->oxaloacetate+ADP+Pi
Similarly, adenylate cyclase:
ATP<->cyclic AMP+PPi
asparagine synthetase:
L-aspartate+ATP<->L-asparagine+AMP+PPi
And succinyl-CoA-synthetase:
Succinate+CoA-SH+ATP<->succinyl-CoA+ADP+Pi
And gamma-glutamylcysteine synthetase:
L-glutamate+L-cysteine+ATP<->L-gamma-glutamylcysteine.
In each case, the result of the reaction is conversion of a nucleoside triphosphate to a di- or mono-phosphate, the desired result as described above.

The enzymes selected for the reactions described above are selected according to their ability to be at least 80% denatured, more preferably 90%, more preferably 95% denatured at a temperature of less than 90° C. in 20 minutes or less as determined by reconstitution experiments in which reagents are added to the denatured enzymes and products measured.

Mixtures, Compositions and Kits:

The enzyme and acceptor components described in the present embodiments can be applied separately to the amplification reaction mixture. That is, individual elements of phosphate-transferring enzyme(s), acceptor(s) and nuclease(s) can be added in separate reactions, using appropriate buffers in each instance to maximize the desired outcomes. In a preferred embodiment, all necessary enzymes, buffers and reactants can be mixed together in a single, stable storage mixture and added in one step to the amplification mixture. For purposes of a kit, instructions are included with the reagents that may be provided in a mixture or in separate reaction vessels.

The following examples establish that nucleotide depletion can be readily achieved by enzymes other than phosphatases involved in nucleic acid metabolism in addition to acceptors. These enzyme/acceptors have the advantage of being capable of heat denaturation.

### EXAMPLES

### Example I

### Cloning, Expression and Purification of PPGK

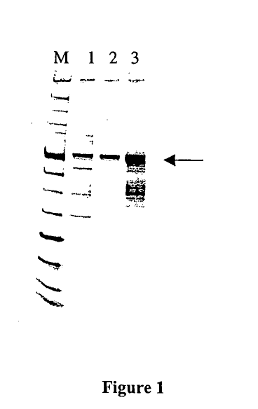

Two primers 5′ ATGACCAGCACCGGCCCCGAGACGTC 3′ (SEQ ID NO:1) and 5′ TATGGATCCTCAGTGCGTCGTATCTGCGACAGAGGCC 3′ (SEQ ID NO:2) were designed to amplify PPGK (GI: 31791177) from Mycobacteria tuberculosis genomic DNA (ATCC 19015D) using PCR. The amplified fragment was digested with BamHI and cloned into pMAL-c2x vector (New England Biolabs, Inc., Ipswich Mass., catalog #N8076) cut with XmnI and BamHI. The fusion protein encoded by this construct was expressed in an Escherichia coli host and purified to apparent homogeneity by amylose affinity chromatography using recommendations given by the supplier (New England Biolabs, Inc., Ipswich, Mass.). The purified enzyme was then dialysed against 50 mM glucose, 50% glycerol, 10 mM MgCl2, 1 mM EDTA and 1 mM β-mercaptoethanol. The purified enzyme is shown in FIG. 1. From 10 ml LB culture, approximately ˜0.5-1 mg of MBP-polyphosphate glucokinase fusion protein was obtained.

### Example II

### PPGK can Utilize dNTP Substrates in Phosphate Transfer

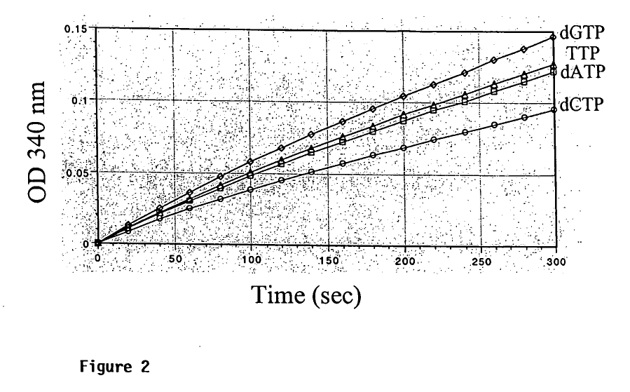

The enzymatic activity of PPGK was determined in a coupled assay that monitored spectroscopically the formation of NADH (FIG. 2). This assay employed the coupled simultaneous reaction catalyzed by glucose-6-phosphate dehydrogenase to trace the appearance of the end product of the PPGK reaction, glucose-6-phosphate:
α-D-Glucose 6-P+NAD->D-6-P-glucono-δ-lactone+NADH (Reaction 7).
Under the assay conditions, transfer of the gamma-phosphate from the substrate dNTP is linked to the formation of NADH, thus NADH production is a measure of the conversion of dNTP to dNDP by PPGK. NAD and NADH can be distinguished spectroscopically on the basis of different extinction coefficients at 340 nm.

The coupled assay was used to test the ability of PPGK to transfer the gamma phosphate of each of the four dNTPs to glucose-6-P. Each nucleotide was assayed individually in reactions containing 50 mM TrisHCl (pH 8.0), 50 mM glucose, 80 mM NaCl, 10 mM MgCl2, 0.5 mM NAD, 1 unit glucose-6-phosphate dehydrogenase and dNTPs (4 mM dATP, dCTP or TTP, or 2 mM dGTP). The appearance of NADH was monitored spectroscopically at 340 nm.

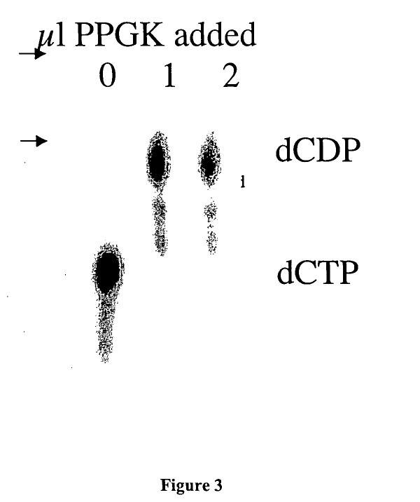

As can be seen in FIG. 2, each of the four dNTPs are substrates for the PPGK reaction, and have similar kinetics of phosphate transfer. The transfer efficiency of the gamma-phosphate from dNTPs to glucose in a mixture typical of amplification was tested using a radioactive assay. A mock PCR reaction was created, containing 10 mM TrisHCl (pH 8.3), 50 mM KCl, 1.5 mM MgCl2, 0.01% gelatin, 1 μg/ml pBR322 DNA, 0.1 mM dNTPs (each nucleotide), 0.5 μM New England Biolabs, Inc., Ipswich, Mass., primer #1239, 0.5 μM New England Biolabs, Inc., Ipswich, Mass., primer #1240 and 0.016 μM α-[³²P]-dCTP (400 Ci/mmole). To 45 μl of this mixture was added 5 μl of PPGK 10× buffer (0.5 M glucose, 0.1 M MgCl2, 1 M NaCl). To this mixture was added 0, 1.5 or 3 μg of PPGK, followed by incubation at 37° C. for 15 minutes. Products were spotted on a polyethylene-imine plate and reaction products were separated by ascending thin layer chromatography: 0.5 minutes using 0.5 M sodium formate (pH 3.4), 2 minutes using 2 M sodium formate (pH 3.4), followed by 4 M sodium formate (pH 3.4) until the solvent front had traveled approximately 12 cm. (Tjaden, et al. J. Biol. Chem. 273:9630-9636 (1998)). Plates were then dried and exposed for about 15 minutes to a K screen, and visualized using a phosphoimager (Bio-Rad, Hercules, Calif.; FIG. 3). Lanes are labeled in this Figure according to the number of μg of PPGK enzyme added to the reaction. The starting material, dCTP, appears to be completely degraded by this assay, as shown by the absence of a spot corresponding to dCTP after enzyme treatment (lanes marked 1 and 2).

Similar experiments with dATP and TTP demonstrated that they too were substrates for the enzyme.

### Example III

### Heat Inactivation of PPGK and Exonuclease I

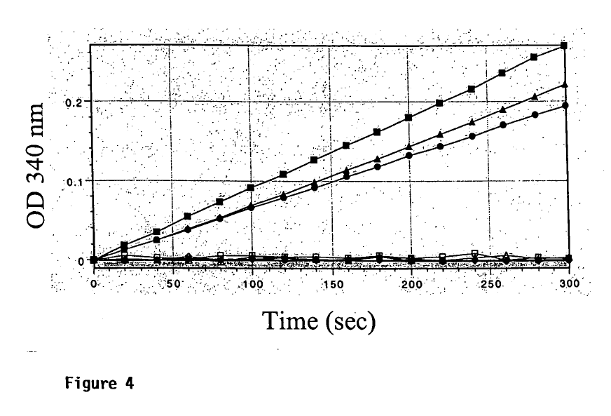

PPGK (1.5 μg) was incubated in 200 μl of (a) 50 mM TrisHCl (pH 8.0), 5 mM MgCl2 or (b) New England Biolabs, Inc., Ipswich, Mass. Thermopol buffer (Catalog #9004) for 15 minutes at 80° C. or at 4° C. Following this incubation, samples were assayed using the coupled assay described in Example II (FIG. 4). No increase in absorbance at 340 nm was noted in heated samples, indicating heat treatment completely inactivated PPGK.

### Example IV

### Use of PPGK to Deplete dNTP Pools

To show the potential for PPGK to deplete dNTP pools, a mock amplification reaction was set up, including trace amounts of α-[³²P]-dATP. The α-[³²P]-dADP product was separated from the initial substrate using thin-layer chromatography as described in Example II. The relative amounts of both species were then determined.

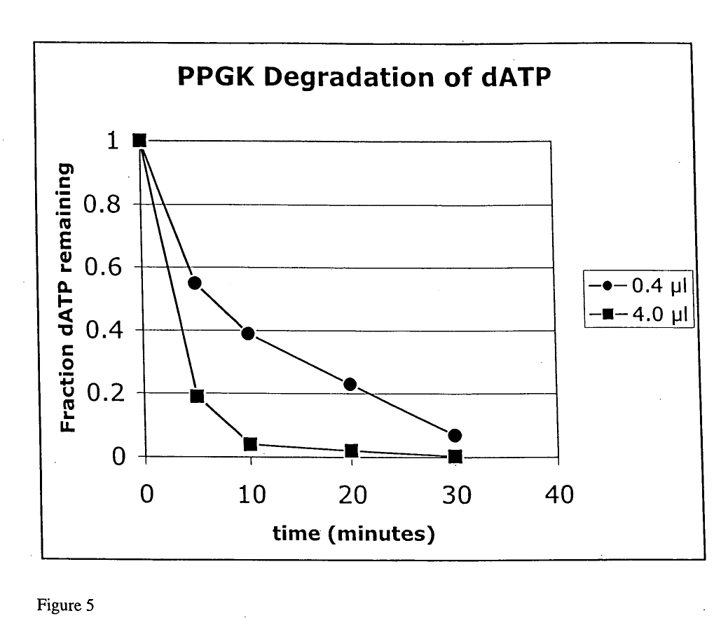

Reactions contained 1× Thermopol buffer (New England Biolabs, Inc., Ipswich, Mass.: 10 mM KCl, 20 mM TrisHCl (pH 8.8 @ 25° C.), 10 mM (NH₄)₂SO₄, 2 mM MgSO₄, 0.1% Triton X-100), 1 μg/ml pBR322 plasmid DNA (New England Biolabs, Inc., Ipswich, Mass.), 0.5 μM oligonucleotide primer S1205S (New England Biolabs, Inc., Ipswich, Mass.), 0.5 μM oligonucleotide primer S1240S, 0.4 mM dNTPs (concentration of each dNTP, 1.6 mM total dNTPs, New England Biolabs, Inc., Ipswich, Mass.), 50 mM D-glucose, 80 mM NaCl, 2 mM MgCl₂, 0.012 μM α-[³²P]-dATP (specific activity approximately 1500 Ci/mmol). Reactions were initiated by addition of PPGK, either 0.4 μl, or 4.0 μl of a 1.5 mg/ml stock, in a reaction volume of 40 μl. At indicated times, a 1 μl aliquot was removed from the reaction and spotted on a PEI thin layer chromatography plate, which was then developed by ascending chromatography with a 0.35 M LiCl solution. After drying, the plate was exposed to a phosphoimager K screen (BioRad, Hercules, Calif.), and quantified using a phosphoimager (BioRad, Hercules, Calif.) and accompanying Quantity One software (BioRad, Hercules, Calif.) (FIG. 5).

Essentially all of the dATP was converted to dADP over the 30 minute time course of the assay with the higher concentration of enzyme. Separate experiments with alternate deoxynucleotides, i.e. dCTP and TTP, suggest that they too can be depleted using similar reaction conditions.

### Example V

### Use of a Coupled Enzyme System to Deplete dNTPs (Nucleoside-5′-Diphosphate Kinase and Hexokinase)

To show the potential for the combined enzymes NDPK and hexokinase to deplete dNTP pools, a mock amplification reaction was set up, including trace amounts of α-[³²P]-dCTP. The α-[³²P]-dCDP product was separated from the initial substrate using thin-layer chromatography as described in Example II. The relative amounts of both species were then determined.

Reactions contained 1× Thermopol buffer (New England Biolabs, Inc., Ipswich, Mass.: 10 mM KCl, 20 mM TrisHCl (pH 8.8 @ 25° C.), 10 mM (NH₄)₂SO₄, 2 mM MgSO₄, 0.1% Triton X-100), 1 μg/ml pBR322 plasmid DNA (New England Biolabs, Inc., Ipswich, Mass.), 0.5 μM oligonucleotide primer S1205S (New England Biolabs, Inc., Ipswich, Mass.), 0.5 μM oligonucleotide primer S1240S, 0.4 mM dNTPs (concentration of each dNTP, 1.6 mM total dNTPs, New England Biolabs, Inc., Ipswich, Mass.), 0.004 μM α-[³²P]-dCTP (specific activity approximately 1500 Ci/mmol, PKI). To 20 μl of this mixture was added 2 μl of the following enzyme mixture: 50% glycerol, 10 mM TrisHCl (pH 7.6 at RT, 50 mM), 40 mM D-glucose, 10 mM ADP, 80 units/ml hexokinase (Sigma, St. Louis, Mo., Type F-300), 80 units/ml NDPK (Sigma, St. Louis, Mo., from Bakers Yeast). Reactions were incubated at 37° C. At indicated times, a 1 μl aliquot was removed from the reaction and spotted on a PEI thin layer chromatography plate.

Heat inactivation of the enzyme mixture was evaluated by heating the above reaction mixture, after sampling the final aliquot at 15 minutes, at 80° C. for 15 minutes. The reaction mixture was cooled on ice, and an additional aliquot of α-[³²P]-dCTP was added to the mixture. Once again, samples were taken at 1, 5, 10 and 15 minute time points, spotted on the PEI plate. The nucleotide components were then separated by ascending chromatography using 0.35 M LiCl (pH 7.2).

After drying, the PEI plate was exposed to a phosphoimager K screen (BioRad, Hercules, Calif.), and quantified using a phosphoimager (BioRad, Hercules, Calif.) and accompanying Quantity One software (BioRad, Hercules, Calif.).

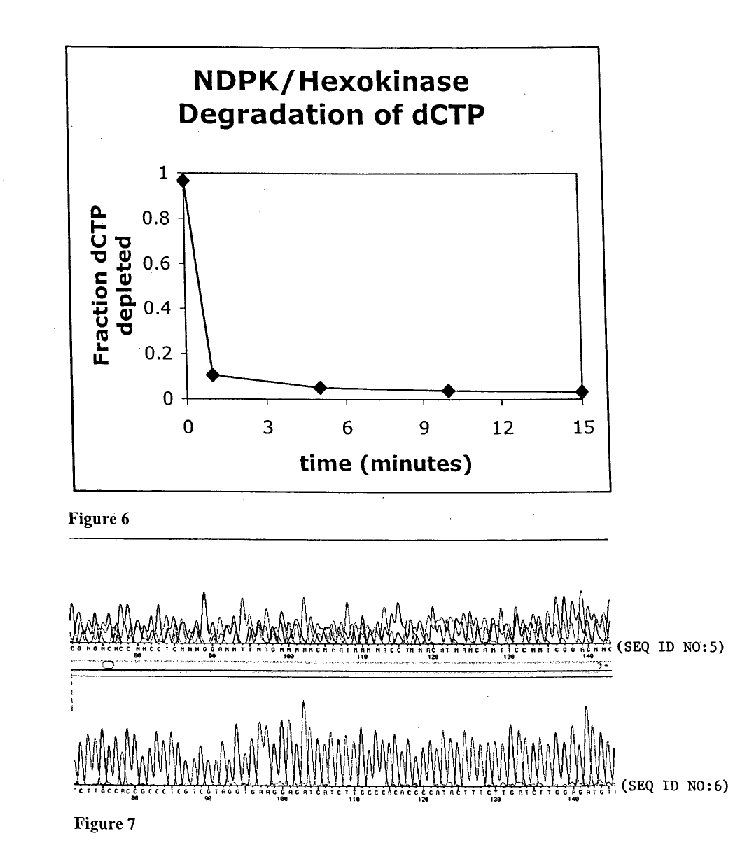

As can be seen in FIG. 6, the dCTP pools were rapidly depleted under these conditions. No further depletion of the nucleotide pool was noted after heat treatment of the sample.

### Example VI

### Depletion of dNTP Pools: Effects on Subsequent Reactions

A PCR reaction, performed in 1× Thermopol buffer (New England Biolabs, Inc., Ipswich, Mass.) using 0.1 mM dNTPs and 0.5 μM of each of two amplification primers, yielded approximately 20 μg/ml of product. 8 μl of this product was mixed with 2 μl of Exonuclease I/PPGK mixture (0.275 M D-glucose, 0.05 M MgCl2, 10% glycerol, 5 U/μl Exonuclease I (New England Biolabs, Ipswich, Mass.), 0.375 μg/μl PPGK) or water and incubated for 20 minutes at 37° C., followed by heating to 80° C. for 20 minutes to inactivate these enzymes. Samples were diluted three-fold into water and submitted for standard sequencing using an ABI sequencer. The top panel of FIG. 7 presents the resulting sequencing trace for the sample treated with water, while the bottom panel is for the sample treated with Exonuclease I and PPGK. Significantly less background signal was observed after treatment with the two enzyme cocktail.

### Example VII

### Depletion of dCTP: Effects on SNP Analysis

As described above, the PCR samples after depletion of dNTPs and primers by PPGK/Exonuclease I mixture could be directly sequenced; alternatively, these samples could be used for detection of SNPs using AcycloPrime-FP SNP Detection Kit G/C (from Perkin Elmer Life Sciences, Inc., Boston, Mass.)). For example, varying amounts of PPGK in a volume of 1 μl (6 ug, 3 ug, 1.5 ug, 0.75 ug, 0.375 ug, 0.18 ug, or 0 ug), 1 ul of supplement buffer (50 mM NaCl and 300 mM Glucose), and 5 ul of 200 uM dCTP, 10 mM TrisHCl (pH 8.3 at 20° C.), 50 mM KCl, 1.5 mM MgCl₂, 20 nM of DNA template (ATTGGATTATTTGTAACTCCAAGGATAAGTGCATAAGGGG) (SEQ ID NO:3), were mixed and incubated together at 37° C. for 15 min. PPGK was then heat inactivated by incubation at 80° C. for 15 min. To this reaction was added 13 ul of AcycloPrime Mix containing 5 pmoles of SNP primer CCCCTTATGCACTTATCCTT (SEQ ID NO:4). Samples were then heated to 95° C. for 2 minutes, and then subjected to 25 cycles of alternate incubation at 95° C. for 1 minute 15 seconds and incubation at 55° C. for 30 seconds. A final incubation at 15° C. for 2 minutes completed the reaction. The incorporation of acyclo terminators was assessed using a PerkinElmer VICTOR 96-well fluorescence polarization detection instrument (PerkinElmer, Boston, Mass.). Control reactions were performed by omitting PPGK and varying the concentration of dCTP in the initial reaction. Results of both sets of reactions are summarized in Table X, with columns 1 and 2 indicating results from PPGK reactions, and columns 3 and 4 indicating results from control reactions titrating dCTP.

In this experiment, higher values in columns labeled “TAMRA 54 (F-dCTP)” indicate the expected incorporation at the SNP site. Control reactions in columns 3 and 4 indicate that dCTP levels must be reduced to at least 1.5 μM in order to obtain an adequate signal. These signal levels are reached when at least 1.5-3 μg of PPGK in included in the reaction mixture.

TABLE X

| | Column 2 | | Column 4 |
|---|---|---|---|
| Column 1 | TAMRA 54 | Column 3 | TAMRA 54 |
| PPGK (ug) | (F-dCTP) | [CTP] uM | (F-dCTP) |
| 6 | 125 | 200 | 2 |
| 3 | 148 | 100 | 9 |
| 1.5 | 135 | 50 | 1 |
| 0.75 | 21 | 12.5 | 32 |
| 0.375 | 11 | 6.25 | 52 |
| 0.18 | 14 | 3.125 | 72 |
| 0 | 13 | 1.5 | 113 |
| | | 0 | 142 |

What is claimed is:

1. A method of depleting a nucleotide pool, comprising:
(a) adding to the nucleotide pool a primary phosphate acceptor and a phosphate-transferring enzyme; and
(b) permitting the conversion of deoxynucleoside triphosphate (dNTP) or ribonucleotide triphosphate (NTP) to a diphosphate so as to deplete the nucleotide pool.

2. A method according to claim 1, wherein the phosphate-transferring enzyme is selected from the group consisting of a nucleoside kinase and a polyphosphate glucokinase.

3. A method according to claim 1, wherein the phosphate-transferring enzyme is a nucleoside kinase and step (a) further comprises a second enzyme selected from a phosphotransferase and a lyase, where the secondary enzyme dephosphorylates the primary phosphate acceptor so as to modify the equilibrium of the reaction with the primary enzyme in favor of dephosphorylation of the dNTP or NTP in the nucleotide pool.

4. A method according to claim 3, wherein the nucleoside kinase is nucleoside 5′diphosphate kinase (NDPK) and the second enzyme is a phosphotransferase.

5. A method according to claim 4, further comprising a secondary phosphate acceptor.

6. A method according to claim 1, wherein the primary enzyme is polyphosphate glucokinase.

7. A method according to claim 1, wherein step (b) further comprises heat inactivating the primary enzyme.

8. A method according to claim 1, wherein pool depletion is at least 85%.

9. A reaction mixture for depleting a nucleotide triphosphate pool, comprising: a gamma phosphate-transferring enzyme for removing a phosphate from a dNTP or NTP in a nucleotide pool; a phosphotransferase or lyase; and a primary nucleoside phosphate acceptor, wherein the phosphotransferase or lyase catalyzes removal of the phosphate from the primary nucleoside phosphate acceptor so as to drive the equilibrium reaction catalyzed by the nucleoside kinase toward depletion of the nucleotide pool.

10. A reaction mixture according to claim 9, wherein the mixture comprising a phosphate-transferring enzyme further comprises a second acceptor.

11. A reaction mixture according to claim 9, wherein the phosphate-transferring enzyme is a nucleoside kinase.

12. A reaction mixture according to claim 11, wherein the nucleoside kinase is NDPK.

13. A reaction mixture according to claim 9, wherein the primary phosphate acceptor molecule is a dNTP or a ribonucleoside diphosphate.

14. A reaction mixture according to claim 13, wherein the primary phosphate acceptor is ADP.

15. A reaction mixture according to claim 9, further comprising a nuclease.

16. A nucleotide depletion reagent capable of gamma phosphate transfer from a dNTP or NTP to a phosphate acceptor so as to reduce the concentration of dNTPs or NTPs in the pool by at least 85%, at least 80% of the depletion reagent being denatured at a temperature of less than 100° C. for an incubation period of less than 60 minutes.

17. A nucleotide depletion reagent according to claim 16, comprising: a nucleoside kinase or a polyphosphate glucokinase and a primary acceptor.

18. A nucleotide depletion reagent according to claim 16, wherein the nucleoside kinase is a NDPK and the reagent further comprises a phosphate transferase and a secondary acceptor.

19. A nucleoside depletion reagent according to claim 16, wherein the nucleoside kinase is a NDPK and the reagent further comprises a lyase.

20. A kit comprising a nucleotide depletion reagent according to claim 9, and optionally instructions for depleting a nucleotide pool.

21. A kit comprising a reaction mixture according to claim 16, and optionally instructions for depleting a nucleotide pool.


SEQUENCE LISTING (6 sequences)

SEQ ID NO: 1 - DNA, 26 bp, unknown
  primer
atgaccagcaccggccccgagacgtc

SEQ ID NO: 2 - DNA, 37 bp, unknown
  primer
tatggatcctcagtgcgtcgtatctgcgacagaggcc

SEQ ID NO: 3 - DNA, 40 bp, unknown
  synthetic DNA template
attggattatttgtaactccaaggataagtgcataagggg

SEQ ID NO: 4 - DNA, 20 bp, unknown
  primer
ccccttatgcacttatcctt

SEQ ID NO: 5 - DNA, 73 bp, unknown
  MI3mpI8
  misc_feature (3)..(3): n is a, c, g, or t
  misc_feature (5)..(5): n is a, c, g, or t
  misc_feature (7)..(7): n is a, c, g, or t
  misc_feature (10)..(11): n is a, c, g, or t
  misc_feature (16)..(18): n is a, c, g, or t
  misc_feature (22)..(23): n is a, c, g, or t
  misc_feature (26)..(26): n is a, c, g, or t
  misc_feature (29)..(31): n is a, c, g, or t
  misc_feature (33)..(33): n is a, c, g, or t
  misc_feature (35)..(35): n is a, c, g, or t
  misc_feature (39)..(42): n is a, c, g, or t
  misc_feature (47)..(48): n is a, c, g, or t
  misc_feature (53)..(53): n is a, c, g, or t
  misc_feature (55)..(55): n is a, c, g, or t
  misc_feature (58)..(58): n is a, c, g, or t
  misc_feature (63)..(64): n is a, c, g, or t
  misc_feature (71)..(72): n is a, c, g, or t
cgngncnccnncctcnnnggannttntgnnnancnaatnnnntcctnnacatnancanttccnntcggacnnc

SEQ ID NO: 6 - DNA, 74 bp, unknown
  MI3mpI8 pre-treated with Exonuclease I and PPGK
cttgccaccgccctcgtcgtaggtgaaggagatcatcttgcccacacgccatactttcctgatcttggagatgt